(19) United States
(12) Plant Patent Application Publication
(10) Pub. No.: US 2002/0188980 P1
(43) Pub. Date: Dec. 12, 2002

(54) Chrysanthemum plant named 'GEDI ONE SAP'

(76) Inventor: Dirk Pieters, Staden (BE)

Correspondence Address: KNOBBE MARTENS OLSON & BEAR LLP, 2040 MAIN STREET, FOURTEENTH FLOOR, IRVINE, CA, 92614, US

(21) Appl. No.: 09/873,494

(22) Filed: Jun. 4, 2001

(51) Int. Cl. A01H 5/00

(52) U.S. Cl. PLT/290

(57) ABSTRACT

A new and distinct Chrysanthemum plant variety characterized by a decorative type inflorescence, consistent flowering response to short days, blooming consistently after 49 days of short day length, free branching habit, bronze ray florets, very uniform round growth habit, and a large quantity of blooms per flowering branch.

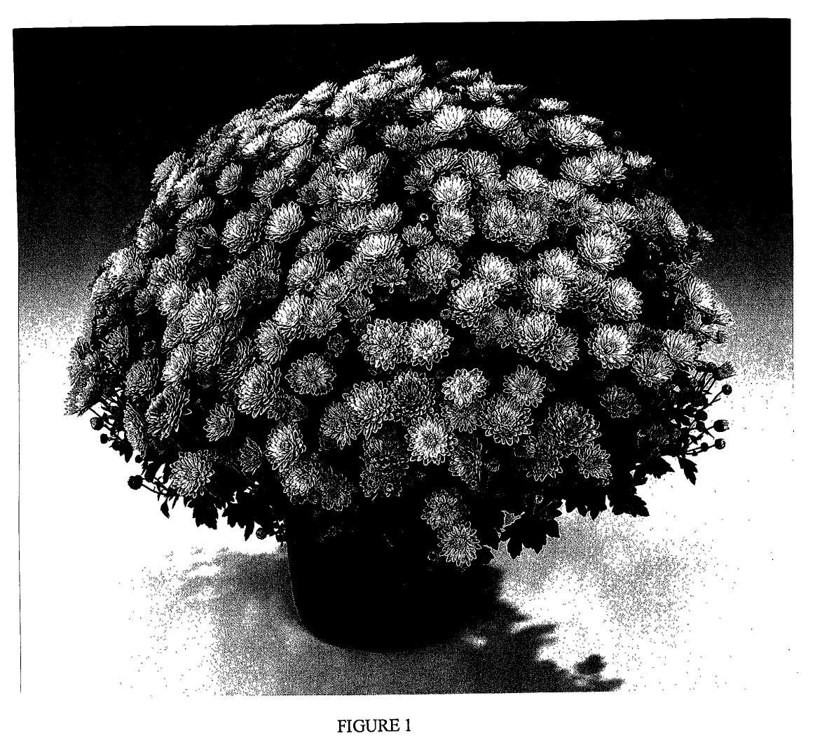

### BACKGROUND OF THE INVENTION

The present invention relates to a new and distinct cultivar of Chrysanthemum plant, botanically known as Dendranthema morifolium and herein after referred to by the cultivar name ‘GEDI ONE SAP.’

This new cultivar was the result of hybridization of a female parent, ‘Cobra’(U.S. Plant Pat. No. 10,219) with a male parent, ‘Pink Elani’(unpatented). The new cultivar was discovered by the inventor in October 1995.

Asexual reproduction of the new cultivar by apical tip cutting and meristem tissue culture was performed in Oxnard, CA and has shown that the unique features of this new cultivar are stable and reproduced true to type on successive generations.

### SUMMARY OF THE INVENTION

The cultivar ‘GEDI ONE SAP’ has not been observed under all possible environmental conditions. The phenotype may vary somewhat with variations in environment such as temperature, daylength, and light intensity, without, however, any variance in genotype.

The following traits have been repeatedly observe and are determined to be the unique characteristics of ‘GEDI ONE SAP.’ These characteristics in combination distinguish ‘GEDI ONE SAP’ as a new and distinct Chrysanthemum:

1. Decorative type inflorescence;

2. Consistent flowering response to short days, blooming consistently after 49 days of short day length;

3. Free branching habit;

4. Bronze ray florets;

5. Very uniform round growth habit; and

6. Large quantity of blooms per flowering branch.

Plants of the new Chrysanthemum are similar to plants of the parent cultivars in most horticultural characteristics, however plants of the new cultivar differ from plants of the female parent cultivar ‘Cobra’ in the following characteristics:

1. The new cultivar has more blooms per flowering branch;

2. The new cultivar naturally blooms 3 weeks later;

3. The new cultivar has a different bloom type; and

4. The new cultivar has smaller diameter flowers.

The new cultivar also differed from the male parent ‘Pink Elani’ in the following characteristics:

1. The new cultivar has an different ray color; and

2. The new cultivar has a different flower type.

The new cultivar can be compared to the Chrysanthemum cultivar ‘Aldo’ (disclosed in Plant Patent No. 10,216). However plants of the new Chrysanthemum differed from the plants of the cultivar ‘Aldo’ in the following characteristics:

1. New cultivar naturally blooms approximately 2 weeks later;

2. New cultivar has more ray florets;

3. New cultivar has a more round plant shape and greater branching; and

4. New cultivar has more blooms per flowering branch.

### BRIEF DESCRIPTION OF THE PHOTOGRAPHS

The photograph shows a plant of ‘GEDI ONE SAP’ grown in a 2-gallon container. A single cutting was used in the container. It was planted in mid-May and grown under outdoor field conditions.

### DETAILED BOTANICAL DESCRIPTION

In the following description, color references are made to the Royal Horticultural Society Colour Chart except where general terms of ordinary dictionary significance are used. The following observations and measurements describe plants grown in a commercial filed on Oxnard, CA in the month of September 2000. The growing conditions range from 60°to 65° F. at night to 70° to 85°during the day. Measurements and numerical values represent averages of typical flowering types. Botanical classification: Dendranthema morifolium cultivar ‘GEDI ONE SAP’ Commercial classification: Garden-type Chrysanthemum Parentage: Hybridization of female parent ‘Cobra2 and male parent ‘Pink Elani’

PROPAGATION

Time to Rooting.—7 to 14 days at approximately 21° C.

Rooting habit.—Fine, fibrous

PLANT

Growth Habit.—Mounding herbaceous perennial

Height.—Approximately 22 cm

Growth rate.—Moderate

Branching Characteristics.—Free Branching

Length of Lateral Branches.—Approximately 24cm

Number of leaves per lateral branch.—Approximately 27

Stem color.—Near 137A

FOLIAGE

Leaf.—Arrangement: Alternate single Average Length: Approximately 6.8 cm Average Width: Approximately 3.9 cm Shape of blade: Ovate Apex: Acuminate Base: Attenuate Attachment: Stalked Margin: Serrate Texture of top surface: Lightly pubescent Texture of bottom surface: Lightly pubescent Color: youngfoliage upper side: Near 137A young foliage under side: Near 137A mature foliage upper side: Near 137A mature foliage under side: Near 137A venation upper side: Near 137C venation under side: Near 137C

Petiole.—Average Length: Approximately 2.5 cm

BLOOM

Inflorescence.—Flowering habit: Induced by darkness period greater than 13.5 hours, approximately 49 days of appropriate day length required to induce and develop blooms Inflorescenceform: Decorative Natural flowering season: Approximately the first two weeks of October Number ofinflorescences per stem: Approximately 17 Inflorescence diameter: Approximately 5.1 cm Inflorescence depth: 1.5 cm Inflorescence longevity on plant: Approximately 22-26 days Persistence: Persistent Ray Florets: Appearance: Matte Texture: Smooth Average Number per flower: 200 Shape: Oblanceolate Margin: Entire Apex: Obtuse Length: Approximately 2.8 cm Width: Approximately 0.8 cm Color: upper surface atfirst opening: Near 168B upper surface at maturity: Near 24B upper surface atfading: Near 23C under surface atfirst opening: Near 25B under surface at maturity: Near 26A under surface atfading: Near 23B

Disc florets.—Appearance: Shiny Texture: Smooth Average number per flower: Approximately 60 Shape: Cylindric Apex: Obtuse Average Length: Approximately 1 cm Average Width: Approximately 0.2 cm Color: at first opening: Near 2A at maturity: Near 21A at fading: Near 17D

Peduncle.—Length: at terminal end (shortest): Approximately 5 cm at lateral end (longest): Approximately 11 cm Strength: Moderate Color: Near 137C Habit: Erect

Inflorescence bud.—Length: Approximately 1 cm Diameter: Approximately 1 cm Form: Globular Color: Near 168A Sepals: Appearance: Matte Texture: Lightly pubescent Number: Approximately 40 to 50 Shape: Ovate Margin: Entire Apex: Acute Color: upper side: Near 137A underside: Near 137A

REPRODUCTIVE ORGANS

Ray Florets.—Pistillate: Number ofpistils per flower: 1 Stigma shape: 2 branched Stigma color: Near 17A Style color: Near 17D Style length: 0.5 cm

Disc Florets.—Pistillate: Number ofpistils perflower: 1 Stigma shape: Cylindric Style length: 0.5 cm Staminate: Number ofstamens perflower: Approximately 5 Anther shape: Tubular Anther color: Near 16A Pollen color: Near 17B

OTHER CHARACTERISTICS

Disease Resistance.—Neither resistance nor susceptibility observed

‘GEDI ONE SAP’: Table of R.H.S. Color Codes

| | | | |
|---|---|---|---|
| FOLIAGE | Leaf | young foliage upper surface | Near 137A |
| | | young foliage under surface | Near 137A |
| | | mature foliage upper surface | Near 137A |
| | | mature foliage under surface | Near 137A |
| | | venation upper surface | Near 137C |
| | | venation under surface | Near 137C |
| BLOOM | Ray Florets | upper surface at first opening | Near 168B |
| | | upper surface at maturity | Near 24B |
| | | upper surface at fading | Near 23C |
| | | under surface at first opening | Near 25B |
| | | under surface at maturity | Near 26A |
| | | under surface at fading | Near 23B |
| | | Pistillate: Stigma | Near 17A |
| | | Pistillate: Style | Near 17D |
| | Disc Florets | at first opening | Near 2A |
| | | at maturity | Near 21A |
| | | at fading | Near 17D |
| | | Staminate: Anther | Near 16A |
| | | Staminate: Pollen | Near 17B |
| | Peduncle | | Near 137C |
| | Inflorescence | | Near 168A |
| | Bud | | |
| | Sepals | upper surface | Near 137A |
| | | under surface | Near 137A |

What is claimed is:

1. A new and distinct variety of Chrysanthemum plant named ‘GEDI ONE SAP’ as herein illustrated and described.
